(19) United States
(12) Patent Application Publication
(10) Pub. No.: US 2026/0145695 A1
(43) Pub. Date: May 28, 2026

(54) Apparatus and Method for Determining State of Vehicle Occupant

(71) Applicants: Hyundai Motor Company, Seoul (KR); Kia Corporation, Seoul (KR)

(72) Inventors: Dong Gu Kim, Hwaseong-Si (KR); Sang Hyeok Yang, Hwaseong-Si (KR); Jang Hyeon Lee, Hwaseong-Si (KR)

(21) Appl. No.: 19/177,851

(22) Filed: Apr. 14, 2025

(30) Foreign Application Priority Data
Nov. 6, 2024 (KR) 10-2024-0156361

(51) Int. Cl. B60W 50/12; B60W 40/08; B60W 60/00; G01N 21/27; G01N 33/497

(52) U.S. Cl. CPC B60W 50/12; B60W 40/08; B60W 60/0015; G01N 21/27; G01N 33/4972; B60W 2040/0836; B60W 2420/40; B60W 2540/227; B60W 2540/24; B60W 2556/40

(57) ABSTRACT

An apparatus of a vehicle may comprise a scanner configured to scan, using an infrared light source, an area of an occupant in the vehicle. At least a portion of infrared light emitted from the infrared light source may be absorbed by a specific gas in the vehicle. A processor may be configured to generate, based on the at least the portion of infrared light absorbed by the specific gas, a concentration indicator indicating a concentration level of the specific gas. The processor may determine, based on the concentration indicator, a state of the occupant and generate, based on the determined state of the occupant, a control signal configured to restrict at least one operation of the vehicle.

### CROSS-REFERENCE TO RELATED APPLICATION

This application claims the benefit of priority to Korean Patent Application No. 10-2024-0156361, filed in the Korean Intellectual Property Office on Nov. 6, 2024, the entire contents of which are incorporated herein by reference.

### TECHNICAL FIELD

The present disclosure relates to an apparatus configured to determine a state of an occupant of a vehicle and a method thereof, and more particularly, relate to an apparatus configured to determine a state of an occupant of a vehicle using infrared rays, and a method thereof.

### BACKGROUND

The matters described in this Background section are only for enhancement of understanding of the background of the disclosure, and should not be taken as acknowledgment that they correspond to prior art already known to those skilled in the art.

To prevent driving under the influence (e.g., drunk driving, operating a vehicle while under the influence of alcohol, etc.), devices may be built into a vehicle to measure the alcohol concentration level of a driver in the vehicle.

The driver's alcohol concentration level may be obtained by measuring alcohol concentration level in the driver's exhalation by using a respiratory sensor. It may take a certain amount of time for the driver's exhalation to be measured after the exhalation enters the sensor. Accordingly, the driver may feel uncomfortable because it may take time for the vehicle to start after the door is opened and then the driver enters the vehicle.

Moreover, not only the driver's exhalation but also a passenger's exhalation may be incorrectly entered into the respiratory sensor. Accordingly, when a drunken passenger is on board, the exhalation of the drunken passenger may cause the alcohol concentration level to be incorrectly measured, thereby resulting in an additional delay in starting the vehicle.

In some cases, alcohol concentration level may be measured by using the exhalation of a passenger instead of the exhalation of a drunken driver in an attempt to bypass the respiratory sensor. Thus, a separate device for recognizing and monitoring a driver is considered to ensure that the exhalation introduced to the respiratory sensor is verified as coming from the driver.

### SUMMARY

The present disclosure was made to solve the above-mentioned problems.

According to the present disclosure, an apparatus of a vehicle, the apparatus may comprise a scanner configured to scan, using an infrared light source, an area of an occupant in the vehicle, wherein at least a portion of infrared light, emitted from the infrared light source, is absorbed by a specific gas in the vehicle, and a processor configured to generate, based on the at least the portion of infrared light absorbed by the specific gas, a concentration indicator, wherein the concentration indicator indicates a concentration level of the specific gas, determine, based on the concentration indicator, a state of the occupant, and generate, based on the determined state of the occupant, a control signal configured to restrict at least one operation of the vehicle.

The apparatus, wherein the specific gas is at least partially present in exhalation of the occupant.

The apparatus, wherein the processor is configured to set a wavelength range of the infrared light source such that the at least the portion of infrared light is absorbed by the specific gas, and wherein the specific gas is an alcohol gas.

The apparatus, wherein the processor is configured to determine, based on transmittance characteristics of the infrared light passing through the alcohol gas, an absorbance level of the alcohol gas.

The apparatus, wherein the processor is configured to generate, based on the absorbance level of the alcohol gas, the concentration indicator.

The apparatus, wherein the processor is configured to determine, based on the concentration indicator, that the occupant is in an intoxicated state.

The apparatus, wherein the processor is configured to determine, based on the concentration indicator, a location of the occupant in the vehicle.

The apparatus, wherein the processor is configured to determine, based on the location of the occupant in the vehicle, that the occupant is a driver of the vehicle, and allow an ignition of the vehicle to be locked such that the occupant in the intoxicated state is prevented from driving the vehicle.

The apparatus, wherein the processor is configured to, based on a predetermined period expiring while the vehicle is driving, re-determine, based on a newly generated concentration indicator, a state of the occupant, wherein the newly generated concentration indicator indicates a concentration level of the alcohol gas after the predetermined period.

The apparatus, wherein the processor is configured to, based on the re-determined state of the occupant being in an intoxicated state and based on the occupant being a driver of the vehicle, perform a minimum risk maneuver operation to stop the vehicle.

According to the present disclosure, a method performed by an apparatus of a vehicle, the method may comprise scanning, using an infrared light source, an area of an occupant in the vehicle, wherein at least a portion of infrared light, emitted from the infrared light source, is absorbed by a specific gas in the vehicle, generating, based on the at least the portion of infrared light absorbed by the specific gas, a concentration indicator, wherein the concentration indicator indicates a concentration level of the specific gas, determining, based on the concentration indicator, a state of the occupant, and generating, based on the determined state of the occupant, a control signal configured to restrict at least one operation of the vehicle.

The method, wherein the specific gas is at least partially present in exhalation of the occupant.

The method may further comprise setting a wavelength range of the infrared light source such that the at least the portion of infrared light is absorbed by the specific gas, and wherein the specific gas is an alcohol gas.

The method may further comprise determining, based on transmittance characteristics of the infrared light passing through the alcohol gas, an absorbance level of the alcohol gas.

The method may further comprise generating, based on the absorbance level of the alcohol gas, the concentration indicator.

The method may further comprise determining, based on the concentration indicator, that the occupant is in an intoxicated state.

The method may further comprise determining, based on the map, a location of the occupant in the vehicle.

The method may further comprise determining, based on the location of the occupant in the vehicle, that the occupant is a driver of the vehicle, and allowing an ignition of the vehicle to be locked such that the occupant in the intoxicated state is prevented from driving the vehicle.

The method may further comprise while the vehicle is driving, determining, based on a newly generated concentration indicator, a second state of the occupant, wherein the newly generated concentration indicator indicates a concentration level of the specific gas exceeding a threshold, and performing, based on the second state of the occupant and based on the occupant being a driver of the vehicle, a minimum risk maneuver operation to stop the vehicle.

According to the present disclosure, a method performed by an apparatus of a vehicle, the method may comprise emitting light toward a designated area within the vehicle, wherein at least a portion of the light is absorbable by a targeted gas, indicating, based on the at least the portion of the light being absorbed by the targeted gas, a concentration level of the targeted gas, determining, based on the indicating, a condition of an occupant associated with the designated area within the vehicle, and controlling, based on the determined condition of the occupant, at least one operation of the vehicle.

### BRIEF DESCRIPTION OF THE DRAWINGS

The above and other objects, features and advantages of the present disclosure will be more apparent from the following detailed description taken in conjunction with the accompanying drawings:

FIG. 1 shows an example of a configuration of an apparatus configured to determine a state of an occupant of a vehicle, according to an example of the present disclosure;

FIG. 2 shows an example of a configuration of a scanner, according to an example of the present disclosure;

FIG. 3 shows an example of a method for obtaining transmittance of an infrared light source, according to an example of the present disclosure;

FIG. 4 shows an example of transmittance characteristics of an infrared light source when an infrared light source is not absorbed by a specific gas, according to an example of the present disclosure;

FIG. 5 shows an example of transmittance characteristics of an infrared light source when an infrared light source is absorbed by a specific gas, according to an example of the present disclosure;

FIG. 6 shows an example of a scanning method for generating a gas concentration mapping image, according to an example of the present disclosure;

FIG. 7 shows an example of a driver alcohol concentration mapping image of a drunken state, according to an example of the present disclosure;

FIG. 8 shows an example of a passenger alcohol concentration mapping image of a drunken state, according to an example of the present disclosure;

FIG. 9 shows an example of a second-row occupant alcohol concentration mapping image of a drunken state, according to an example of the present disclosure;

FIG. 10 shows an example of a point cloud image determined as not boarding a driver, according to an example of the present disclosure;

FIG. 11 shows an example of a point cloud image determined as boarding a driver, according to an example of the present disclosure;

FIG. 12 shows an example of a guidance guide output according to an example of the present disclosure;

FIG. 13 shows an example of a method for determining a vehicle occupant state before driving, according to an example of the present disclosure;

FIG. 14 shows an example of a method for determining a vehicle occupant state during driving, according to an example of the present disclosure; and

FIG. 15 shows an example of a configuration of a computing system performing a method, according to an example of the present disclosure.

### DETAILED DESCRIPTION

Hereinafter, some examples of the present disclosure will be described in detail with reference to the accompanying drawings. In adding reference numerals to components of each drawing, it should be noted that the same components include the same reference numerals, although they are indicated on another drawing. Furthermore, in describing the examples of the present disclosure, detailed descriptions associated with well-known functions or configurations will be omitted when they may make subject matters of the present disclosure unnecessarily obscure.

In describing elements of an example of the present disclosure, the terms first, second, A, B, (a), (b), and the like may be used herein. These terms are only used to distinguish one element from another element, but do not limit the corresponding elements irrespective of the nature, order, or priority of the corresponding elements. Furthermore, unless otherwise defined, all terms including technical and scientific terms used herein are to be interpreted as is customary in the art to which the present disclosure belongs. It will be understood that terms used herein should be interpreted as including a meaning that is consistent with their meaning in the context of the present disclosure and the relevant art and will not be interpreted in an idealized or overly formal sense unless expressly so defined herein.

For purposes of this application and the claims, using the exemplary phrase “at least one of: A; B; or C” or “at least one of A, B, or C,” the phrase means “at least one A, or at least one B, or at least one C, or any combination of at least one A, at least one B, and at least one C. Further, exemplary phrases, such as “A, B, and C”, “A, B, or C”, “at least one of A, B, and C”, “at least one of A, B, or C”, etc. as used herein may mean each listed item or all possible combinations of the listed items. For example, “at least one of A or B” may refer to (1) at least one A; (2) at least one B; or (3) at least one A and at least one B.

An automation level of an autonomous driving vehicle may be classified as follows, according to the American Society of Automotive Engineers (SAE). At autonomous driving level 0, the SAE classification standard may correspond to “no automation,” in which an autonomous driving system is temporarily involved in emergency situations (e.g., automatic emergency braking) and/or provides warnings only (e.g., blind spot warning, lane departure warning, etc.), and a driver is expected to operate the vehicle. At autonomous driving level 1, the SAE classification standard may correspond to “driver assistance,” in which the system performs some driving functions (e.g., steering, acceleration, brake, lane centering, adaptive cruise control, etc.) while the driver operates the vehicle in a normal operation section, and the driver is expected to determine an operation state and/or timing of the system, perform other driving functions, and cope with (e.g., resolve) emergency situations. At autonomous driving level 2, the SAE classification standard may correspond to “partial automation,” in which the system performs steering, acceleration, and/or braking under the supervision of the driver, and the driver is expected to determine an operation state and/or timing of the system, perform other driving functions, and cope with (e.g., resolve) emergency situations. At autonomous driving level 3, the SAE classification standard may correspond to “conditional automation,” in which the system drives the vehicle (e.g., performs driving functions such as steering, acceleration, and/or braking) under limited conditions but transfer driving control to the driver when the required conditions are not met, and the driver is expected to determine an operation state and/or timing of the system, and take over control in emergency situations but do not otherwise operate the vehicle (e.g., steer, accelerate, and/or brake). At autonomous driving level 4, the SAE classification standard may correspond to “high automation,” in which the system performs all driving functions, and the driver is expected to take control of the vehicle only in emergency situations. At autonomous driving level 5, the SAE classification standard may correspond to “full automation,” in which the system performs full driving functions without any aid from the driver including in emergency situations, and the driver is not expected to perform any driving functions other than determining the operating state of the system. Although the present disclosure may apply the SAE classification standard for autonomous driving classification, other classification methods and/or algorithms may be used in one or more configurations described herein.

One or more features associated with autonomous driving control may be activated based on configured autonomous driving control setting(s) (e.g., based on at least one of: an autonomous driving classification, a selection of an autonomous driving level for a vehicle, etc.). Based on one or more features (e.g., feature of detecting a condition (e.g., intoxicated, drunken, etc.) of an occupant (e.g., driver) in the vehicle) described herein, an operation of the vehicle may be controlled. The vehicle control may include various operational controls associated with the vehicle (e.g., autonomous driving control, sensor control, braking control, braking time control, acceleration control, acceleration change rate control, alarm timing control, forward collision warning time control, etc.).

One or more auxiliary devices (e.g., engine brake, exhaust brake, hydraulic retarder, electric retarder, regenerative brake, etc.) may also be controlled, for example, based on one or more features (e.g., feature of detecting a condition (e.g., intoxicated, drunken, etc.) of an occupant (e.g., driver) in the vehicle) described herein.

One or more communication devices (e.g., a modem, a network adapter, a radio transceiver, an antenna, etc., that is capable of communicating via one or more wired or wireless communication protocols, such as Ethernet, Wi-Fi, near-field communication (NFC), Bluetooth, Long-Term Evolution (LTE), 5G New Radio (NR), vehicle-to-everything (V2X), etc.) may also be controlled, for example, based on one or more features (e.g., feature of detecting a condition (e.g., intoxicated, drunken, etc.) of an occupant (e.g., driver) in the vehicle) described herein.

Biased driving operation(s) may also be controlled, for example, based on one or more features (e.g., feature of detecting a condition (e.g., intoxicated, drunken, etc.) of an occupant (e.g., driver) in the vehicle) described herein. A driving control apparatus may perform a biased driving control. To perform a biased driving, the driving control apparatus may control the vehicle to drive in a lane by maintaining a lateral distance between the position of the center of the vehicle and the center of the lane. For example, the driving control apparatus may control the vehicle to stay in the lane but not in the center of the lane. The driving control apparatus may identify or determine a biased target lateral distance for biased driving control. For example, a biased target lateral distance may comprise an intentionally adjusted lateral distance that a vehicle may aim to maintain from a reference point, such as the center of a lane or another vehicle, during maneuvers such as lane changes. This adjustment may be made to improve the vehicle's stability, safety, and/or performance under varying driving conditions, etc. For example, during a lane change, the driving control system may bias the lateral distance to keep a safer gap from adjacent vehicles, considering factors such as the vehicle's speed, road conditions, and/or the presence of obstacles, etc.

Biased driving operation(s) may also be controlled, for example, based on one or more features (e.g., feature of detecting a condition (e.g., intoxicated, drunken, etc.) of an occupant (e.g., driver) in the vehicle) described herein. A driving control apparatus may perform a biased driving control. To perform a biased driving, the driving control apparatus may control the vehicle to drive in a lane by maintaining a lateral distance between the position of the center of the vehicle and the center of the lane. For example, the driving control apparatus may control the vehicle to stay in the lane but not in the center of the lane. The driving control apparatus may identify or determine a biased target lateral distance for biased driving control. For example, a biased target lateral distance may comprise an intentionally adjusted lateral distance that a vehicle may aim to maintain from a reference point, such as the center of a lane or another vehicle, during maneuvers such as lane changes. This adjustment may be made to improve the vehicle's stability, safety, and/or performance under varying driving conditions, etc. For example, during a lane change, the driving control system may bias the lateral distance to keep a safer gap from adjacent vehicles, considering factors such as the vehicle's speed, road conditions, and/or the presence of obstacles, etc.

One or more sensors (e.g., IMU sensors, camera, LIDAR, RADAR, blind spot monitoring sensor, line departure warning sensor, parking sensor, light sensor, rain sensor, traction control sensor, anti-lock braking system sensor, tire pressure monitoring sensor, seatbelt sensor, airbag sensor, fuel sensor, emission sensor, throttle position sensor, inverter, converter, motor controller, power distribution unit, high-voltage wiring and connectors, auxiliary power modules, charging interface, etc.) may also be controlled, for example, based on one or more features (e.g., feature of detecting a condition (e.g., intoxicated, drunken, etc.) of an occupant (e.g., driver) in the vehicle) described herein. An operation control for autonomous driving of the vehicle may include various driving control of the vehicle by the vehicle control device (e.g., acceleration, deceleration, steering control, gear shifting control, braking system control, traction control, stability control, cruise control, lane keeping assist control, collision avoidance system control, emergency brake assistance control, traffic sign recognition control, adaptive headlight control, etc.).

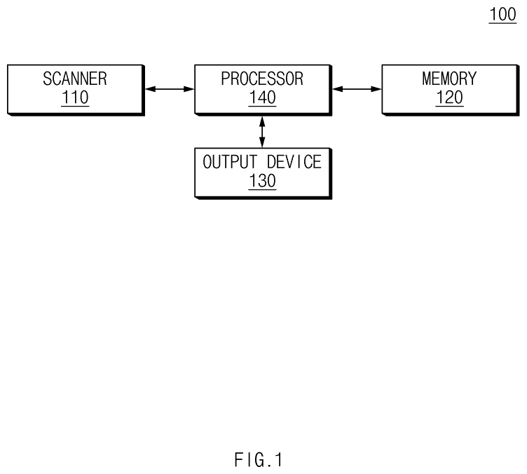

FIG. 1 shows an example of a configuration of an apparatus configured to determine a state of an occupant of a vehicle, according to an example of the present disclosure.

Referring to FIG. 1, an apparatus configured to determine a state of an occupant of a vehicle 100 may include a scanner 110, a memory 120, an output device 130, and a processor 140.

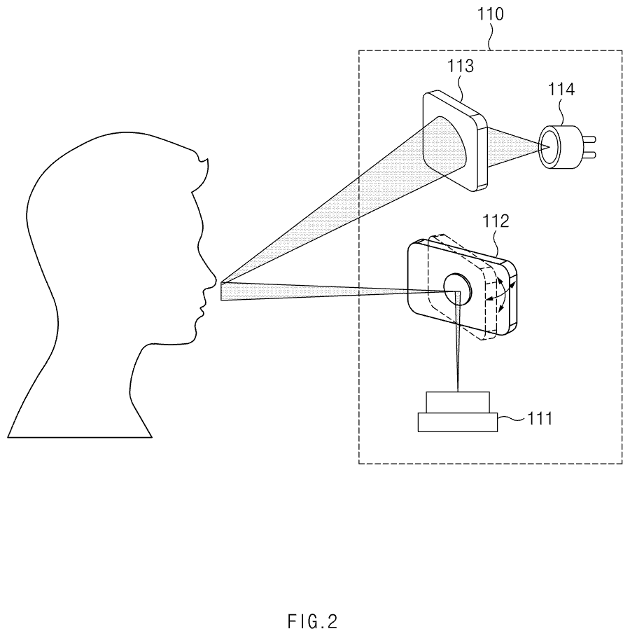

The scanner 110 may scan an occupant area by using an infrared light source absorbed by a specific gas and may obtain the absorbance of the specific gas. The scanner 110 may be located near a steering wheel. Detailed descriptions refer to FIG. 2. Here, the specific gas may include a gas contained within the exhalation of an occupant, and may include, for example, alcohol gas, nitrogen oxide, hydrogen sulfide, carbon monoxide, or the like.

FIG. 2 shows an example of a configuration of a scanner, according to an example of the present disclosure.

As shown in FIG. 2, the scanner 110 (e.g., an optical scanner, a spectroscopic scanner, or a multi-sensor scanning device, etc.) may include a radiation-emitting device (e.g., a light transmitting device 111), a light reflecting device 112 (e.g., a mirror, a prism, or a diffraction grating, etc.), a light receiving lens 113 (e.g., a focusing lens or an optical filter, etc.), and a light receiving device 114 (e.g., a photodetector, an image sensor, or a spectrometer, etc.). The light receiving device 114 may perform a measurement according to a spectroscopy method (e.g., a tunable diode laser absorption spectroscopy) to generate spectrum data that indicates a concentration of target gas(es) (e.g., alcohol gas, nitrogen oxides, or carbon monoxide, etc.).

The light transmitting device 111 may output an infrared light source (e.g., infrared light, ultraviolet light, etc.). According to an example, the infrared light source may include laser light. The light transmitting device 111 may be equipped with a collimating component (e.g., a collimating lens or an optical waveguide, etc.) for directivity of the infrared light source.

The light reflecting device 112 may scan an occupant area or region (e.g., driver seat, passenger seat, or rear seats, etc.) by reflecting the output infrared light source into or toward the occupant area or region. The light reflecting device 112 may rotate in multiple directions (e.g., up, down, left, right, or in a sweeping pattern, etc.) to scan the occupant area or region. Here, the occupant area or region may include a driver's seat, front passenger seats, rear passenger seats, etc.

The light receiving lens 113 may receive or collect the infrared light source reflected from the occupant area or region and direct or guide the reflected infrared light source to the light receiving device 114 for analysis.

The light receiving device 114 may obtain the transmittance of the infrared light source by receiving the infrared light source reflected from the occupant area. The detection device 114 may obtain the transmittance or absorption characteristics of the infrared light source by detecting the infrared light source that has interacted with the occupant region, enabling analysis of substance concentrations (e.g., alcohol gas, nitrogen oxides, carbon monoxide, hydrogen sulfide, or water vapor, etc.).

The memory 120 may store at least one algorithm for processing data (e.g., performing calculation or execution of various commands, etc.) for an operation of an apparatus configured to determine a state of an occupant of a vehicle (e.g., a drunken state, a DUI state, etc.) according to an example of the present disclosure. According to an example, the memory 120 may store at least one instruction executed by the processor 140, and the instruction may cause determining the state of the occupant of the vehicle to operate. The memory 120 may include at least one storage medium of a flash memory, a hard disk, a memory card, a read-only memory (ROM), a random access memory (RAM), an electrically erasable programmable read only memory (EEPROM), a programmable read-only memory (PROM), a magnetic memory, a magnetic disk, an optical disc, or a cloud-based storage unit, etc.

The output device 130 may output video or sound under the control of the processor 140. According to an example, the output device 130 may be implemented as a display device or a sound output device. Here, the display device may include a HUD, a cluster, or the like. According to an example, the display device may be implemented as a display device employing a liquid crystal display (LCD) panel, a light emitting diode (LED) panel, or an organic light emitting diode (OLED) panel. The display device may be implemented as a touch screen panel (TSP) or a voice-interactive screen, etc.

The processor 140 may be implemented by various processing devices (e.g., a central processing unit (CPU), a graphical processing unit (GPU), an application-specific integrated circuit (ASIC), a neural processing unit (NPU), or a field-programmable gate array (FPGA), etc.) equipped with a semiconductor chip capable of performing or executing various commands, and may control an operation of an apparatus configured to determine a state of an occupant of a vehicle according to an example of the present disclosure. The processor 140 may be electrically connected to the scanner 110, the memory 120, and the output device 130 through a wired cable or various circuits to deliver electrical signals including control commands, and to perform calculations or data processing related to control and/or communication. The processor 140 may include at least one of a central processing unit (CPU), an application processor, or a communications processor (CP), or any combination thereof. Alternatively or additionally, the processor 140 may be electrically or wirelessly connected to the scanner 110, the memory 120, and the output device 130 to exchange control commands, perform signal analysis, and manage data processing tasks.

The processor 140 may calculate or determine absorbance characteristics of a specific gas based on the transmittance of the infrared light source obtained or detected by the light receiving device 114. The processor 140 may generate concentration indicator (e.g., a map or a spatially resolved concentration map such as a gas concentration mapping image) based on the absorbance of the specific gas. Here, the absorbance may refer to the extent to which a specific gas absorbs an infrared light source of a specific wavelength or reduces the intensity of the infrared light source at the specific wavelength.

According to an example, the processor 140 may generate a gas concentration mapping image by using the characteristics that some wavelengths of an infrared light source are absorbed by a specific gas or selectively absorbed by different gases.

The processor 140 may set or adaptively configure the wavelength range of the infrared light source differently depending on the type of a specific gas being detected such that the infrared light source is absorbed by the specific gas.

For example, when the infrared light source is absorbed by an alcohol gas (e.g., ethanol, methanol, or isopropanol, etc.), the processor 140 may set the wavelength range of the infrared light source output from the light transmitting device 111 as a first wavelength range (e.g., from 1.3μm or 12 μm, etc.).

For another example, when the infrared light source is absorbed by nitrogen oxide (NOx) (e.g., NO, NO₂, or N₂O, etc.), the processor 140 may set the wavelength range of the infrared light source output from the light transmitting device 111 as a second wavelength range (e.g., from 2.5 μm to 6 μm, etc.). For more examples, for detecting carbon monoxide (CO), the processor 140 may set the infrared light source to emit within a third wavelength range (e.g., from 2.2 μm to 5 μm, etc.). For detecting hydrogen sulfide (H₂S), the processor 140 may set the infrared light source to emit within a fourth wavelength range (e.g., from 1.5 to 10 μm, etc.).

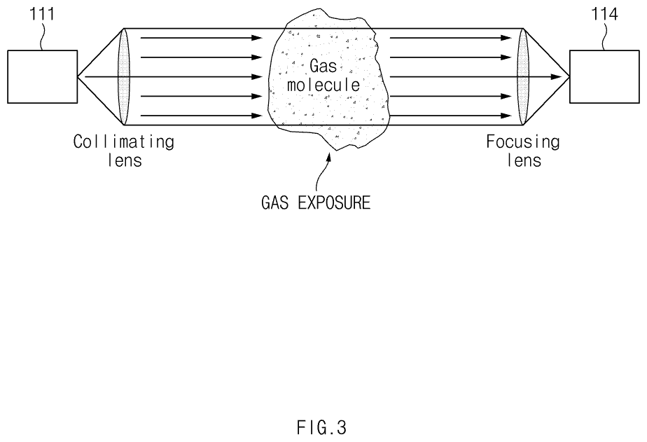

An example of generating a gas concentration mapping image of the present disclosure will be described with reference to FIGS. 3 to 9.

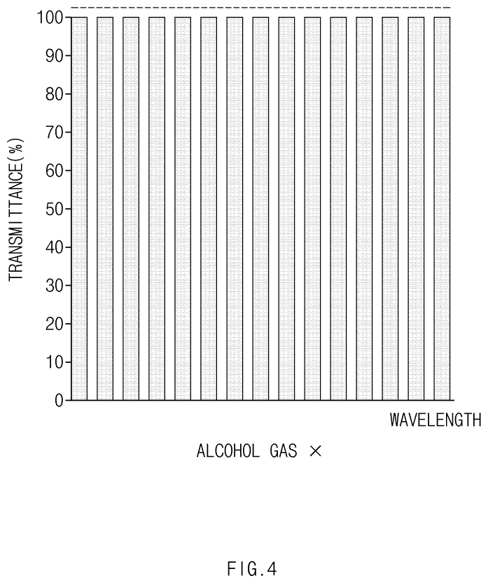

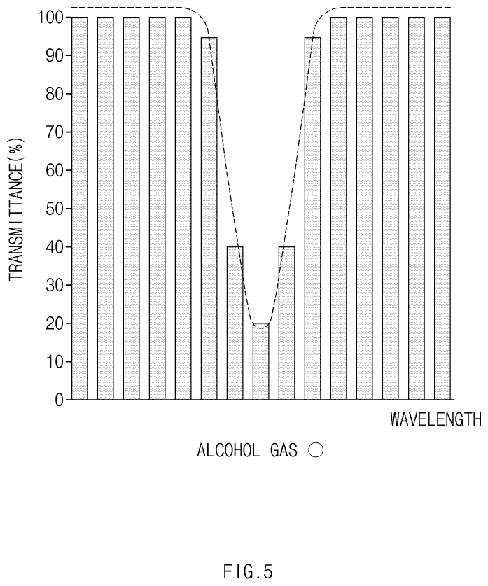

FIG. 3 shows an example of a method for obtaining transmittance characteristics of an infrared light source, according to an example of the present disclosure. FIG. 4 shows an example of transmittance characteristics of an infrared light source when an infrared light source is not absorbed by a target substance (e.g., a specific gas such as alcohol gas, nitrogen oxides, or carbon monoxide, etc.), according to an example of the present disclosure. FIG. 5 shows an example of transmittance variations of an infrared light source when an infrared light source interacts with a target substance (e.g., being absorbed at a specific wavelength range associated with a specific gas), according to an example of the present disclosure. A spectroscopic technique (e.g., Tunable diode laser absorption spectroscopy (TDLAS), differential optical absorption spectroscopy (DOAS), etc.), which is an optical scan method, may be used to obtain the transmittance characteristics of an infrared light source in real-time according to an example of the present disclosure.

As shown in FIG. 3, when the infrared light source output from the light transmitting device 111 passes through a gaseous medium (e.g., a specific gas layer), the infrared light source is absorbed by the specific gas (e.g., ethanol vapor, carbon monoxide, or hydrogen sulfide, etc.) by interacting with the molecules of the specific gas (e.g., partially absorbed by the molecules of the specific gas through photon-molecule interactions). As the infrared light source, which remains after being is absorbed (e.g., remaining portion of the infrared light source, which is not absorbed), is received by the light receiving device 114, the transmittance characteristics of the infrared light source may be obtained.

As shown in FIG. 4, when the infrared light source is not absorbed by a target substance (e.g., a specific gas such as ethanol vapor, carbon monoxide, or hydrogen sulfide, etc.), the infrared light source is transmitted as it is (e.g., without attenuation), and thus there is no change in the transmittance characteristics of the infrared light source depending on the wavelength of the infrared light source. As shown in FIG. 5, when the infrared light source of a predetermined wavelength is absorbed by a specific gas, the transmittance of the infrared light source of a predetermined wavelength decreases (e.g., forming a spectral absorption pattern that is characteristic of the target substance).

The processor 140 may calculate or determine the absorbance characteristics of the specific gas based on the transmittance of an infrared light source (e.g., measured transmittance values). The absorbance characteristics may be determined using spectroscopic analysis techniques (e.g., Beer-Lambert Law, multi-wavelength spectral fitting, or signal deconvolution, etc.) to quantify the concentration of the target substance.

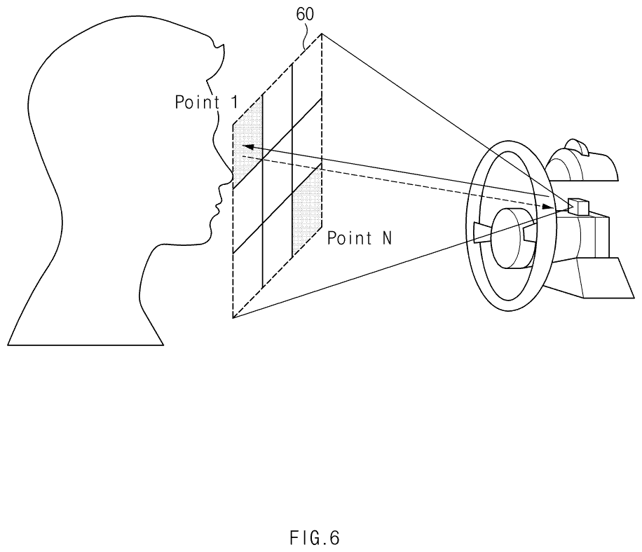

The processor 140 may estimate the concentration level of a target substance (e.g., a specific gas such as ethanol vapor) based on the absorbance characteristics of the specific gas and may generate a spatially resolved concentration map (e.g., a gas concentration mapping image) based on the concentration level of the specific gas. The more detailed description is given with reference to FIGS. 6 to 9.

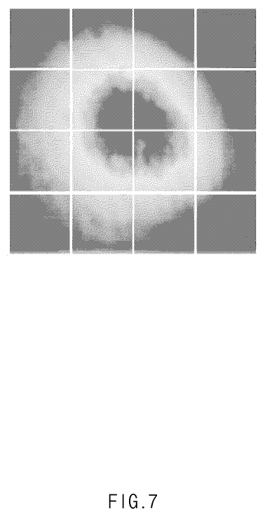

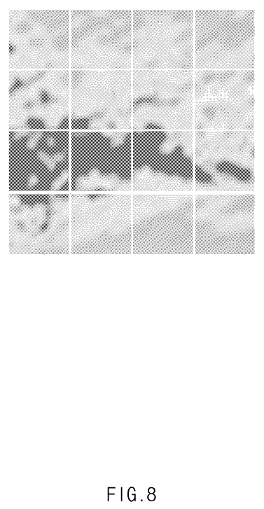

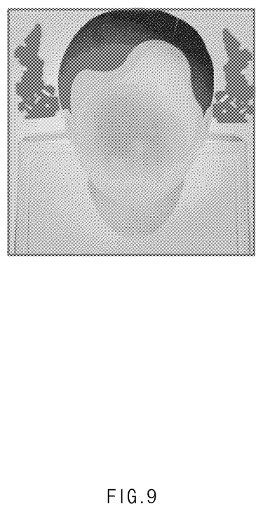

FIG. 6 shows an example of a scanning method (e.g., a multi-point scanning approach) for generating a gas concentration mapping image (e.g., an alcohol concentration map, a CO2 distribution map, or a pollutant detection map, etc.), according to an example of the present disclosure. FIG. 7 shows an example of a driver alcohol concentration mapping image of a drunken state, according to an example of the present disclosure. FIG. 7 may represent an alcohol concentration mapping image generated using infrared absorption characteristics to determine if a driver of a vehicle is intoxicated (e.g., in drunken state). The image may be created by scanning the driver's seat area and mapping alcohol gas concentrations based on infrared absorbance data. FIG. 8 shows an example of a passenger alcohol concentration mapping image of a drunken state, according to an example of the present disclosure. FIG. 9 shows an example of a rear-seat (e.g., a second-row) occupant alcohol concentration mapping image of a drunken state, according to an example of the present disclosure.

As shown in FIG. 6, the processor 140 may control multi-directional movement (e.g., up, down, left, right, rotational, or sweeping motion, etc.) of the light reflecting device 112 to enable scanning of an occupant detection region (e.g., scan an occupant area 60 by reflecting the output light to the occupant area 60). Here, the occupant area 60 may include a predetermined area or predefined scan zones including an occupant face obtainable in a state of sitting in a driver's seat. According to an example, the processor 140 may divide the occupant area 60 into a plurality of scanning zones (e.g., Point 1 to Point N, etc.). The processor 140 may obtain the absorbance characteristics of a specific gas detected for each zone by scanning each zone, and may estimate the gas concentration levels for each zone based on the absorbance of the specific gas. According to an example, the processor 140 may estimate the gas concentration level of a specific gas by using spectroscopic absorption principles such as the Lambert-Beer law or tunable diode laser absorption spectroscopy (TDLAS), etc.

The processor 140 may generate a spatially resolved concentration map (e.g., a gas concentration mapping image) based on the estimated gas concentration level for each scanning zone.

According to an example, the processor 140 may configure the infrared light source to operate within a wavelength range tailored for absorption by a target substance (e.g., alcohol gas). The processor 140 may divide the occupant area into multiple scanning points (e.g., the plurality of zones Point 1 to Point N) and may obtain the absorption data (e.g., for alcohol gas) for each scanned zone. The processor 140 may estimate a concentration level of the target substance (e.g., concentration level of alcohol) for each zone based on the absorption data and generate a concentration map of the target substance (e.g., an alcohol concentration mapping image) according to the estimated concentration level of the target substance. According to an example, the processor 140 may generate a substance concentration map (e.g., the alcohol concentration mapping image) using a color-coded presentation (e.g., a color palette according to the level of an absorbance). For example, the processor 140 may display, in case of an alcohol detection map (e.g., alcohol concentration mapping image, regions with relatively higher alcohol concentration level may be colored in red while relatively low-concentration regions may be colored blue.

The processor 140 may determine whether an occupant is in a drunken state, based on the alcohol concentration mapping image.

According to an example, when the alcohol concentration mapping image is generated as a blue image or predominantly blue in the entire zones of the occupant area, the processor 140 may determine that the occupant is not in a drunken state (e.g., not intoxicated).

According to an example, when a red image or red zones appear in the plurality of zones in the occupant area in the alcohol concentration mapping image, the processor 140 may determine that the occupant is in a drunken state (e.g., intoxicated).

According to an example, the processor 140 may compare a newly generated alcohol concentration mapping image with a reference map (e.g., a pre-stored alcohol concentration mapping image of the occupant of the drunken state) and may determine or identify a location of the occupant of the drunken state. The processor 140 may determine if the newly generated alcohol concentration mapping image matches with one reference image of pre-stored reference images of expected alcohol concentration distributions for different occupant locations. For example, if the newly generated image matches with a reference image of FIG. 7 showing that a relatively high alcohol concentration is centered at the driver's seat, the processor 140 may determine that the driver is intoxicated. If the newly generated image matches with a reference image of FIG. 8 showing that a relatively high alcohol concentration is centered at the front passenger seat, the processor 140 may determine that a passenger is intoxicated. If the newly generated image matches with a reference image of FIG. 9 showing that a relatively high alcohol concentration is centered at the second-row seat, the processor 140 may determine that a rear-seat occupant is intoxicated.

According to an example, if the processor 140 determines that the newly generated alcohol concentration mapping image matches a reference map (e.g., a driver alcohol concentration mapping image of a drunken state, as shown in FIG. 7), the processor 140 may determine that the occupant of the drunken state is the driver.

According to an example, if the processor 140 determines that the newly generated alcohol concentration mapping image matches a reference map of FIG. 8, the processor 140 may determine that the occupant of the drunken state is a passenger. Here, the passenger may include an occupant sitting in a seat next to the driver's seat.

According to an example, if the processor 140 determines that the generated alcohol concentration mapping image matches a reference map of FIG. 9, the processor 140 may determine that the occupant of the drunken state is a second-row occupant. Here, the second-row occupant may include an occupant sitting in a seat located behind the driver's seat.

In addition to detecting alcohol concentration level, in the present disclosure, the processor 140 may generate a substance concentration map (e.g., a gas concentration mapping image) for other gases, such as nitrogen oxide (NOx), hydrogen sulfide (H₂S), or carbon monoxide (CO), and may determine the occupant's sate based on the gas concentration mapping image.

For example, when a specific gas is nitrogen oxide (NOx) (e.g., NO, NO₂, or N₂O, etc.), the processor 140 may configure the infrared light source to emit at wavelength range absorbable by the nitrogen oxide. The processor 140 may divide the occupant area into the plurality of zones Point 1 to Point N, obtain the absorbance of the nitrogen oxide for each zone by scanning each zone. The processor 140 may estimate the nitrogen oxide concentration for each zone based on the absorbance of the nitrogen oxide, and generate a nitrogen oxide concentration mapping image according to the estimated nitrogen oxide concentration. The processor 140 may determine a health-related condition (e.g., asthma) of an occupant based on a nitrogen oxide concentration mapping image.

For example, when a specific gas is hydrogen sulfide (H₂S) (e.g., a biomarker for halitosis or industrial exposure, etc.), the processor 140 may configure the infrared light source to operate at the wavelength range absorbable by the hydrogen sulfide. The processor 140 may divide the occupant area into the plurality of zones Point 1 to Point N, obtain the absorbance of the hydrogen sulfide for each zone by scanning each zone. The processor 140 may estimate the hydrogen sulfide concentration for each zone based on the absorbance of the hydrogen sulfide, and generate a hydrogen sulfide concentration mapping image according to the estimated hydrogen sulfide concentration. The processor 140 may determine a health-related condition (e.g., bad breath or gastrointestinal issues) of an occupant based on the hydrogen sulfide concentration mapping image.

For example, when a specific gas is carbon monoxide (e.g., an indicator of smoking or poor air quality, etc.), the processor 140 may configure the infrared light source to operate at the wavelength range absorbable by the carbon monoxide. The processor 140 may divide the occupant area into the plurality of zones Point 1 to Point N, obtain the absorbance of the carbon monoxide for each zone by scanning each zone. The processor 140 may estimate the carbon monoxide concentration for each zone based on the absorbance of the carbon monoxide, and generate a carbon monoxide concentration mapping image according to the estimated carbon monoxide concentration. The processor 140 may determine a health-related condition (e.g., smoking or exposure to air pollution) of an occupant based on the carbon monoxide concentration mapping image.

When scanning the occupant area as shown in FIG. 6, the processor 140 may determine whether there is an occupant within the occupant area 60.

According to an example, acoustic pulses (e.g., an ultrasonic signal) from an ultrasonic sensor may be transmitted to multiple scanning points distributed throughout the occupant area under the control of the processor 140. The processor 140 may determine a time delay between pulse transmission and echo reception (e.g., a difference between a point in time, when the ultrasonic signal is transmitted, and a point in time, when the ultrasonic signal is received) and determine whether an occupant area is occupied, by using the time delay.

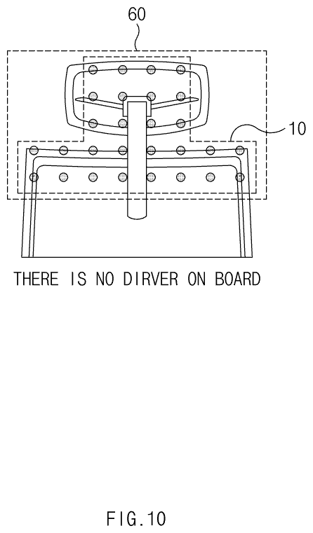

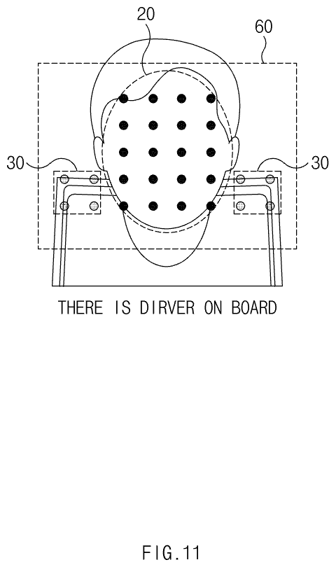

The processor 140 may determine a time delay value for each scanning point distributed throughout the entire occupant area and generate a cluster of points (e.g., point cloud image) based on the time delay value. According to an example, the processor 140 may generate a point cloud image of the same color with respect to points with a similar time delay value. The more detailed description is given with reference to FIGS. 10 and 11.

FIG. 10 shows an example of a point cloud image indicating the absence of a driver, according to an example of the present disclosure. FIG. 11 shows an example of a point cloud image indicating the presence of a driver, according to an example of the present disclosure.

As shown in FIG. 10, when a point cloud dataset (e.g., a structured depth map, a 3D lidar point cloud, or an infrared sensor-generated image, etc.) such as a point cloud image 10 of the same color matches the shape of a vehicle seat, the processor 140 may determine that a driver is not on board.

As shown in FIG. 11, when a point cloud image 20 of the same color matches the central portion of the occupant area 60, and a point cloud image 30 of a different color is generated around the central portion, the processor 140 may determine that the driver is on board. Specifically, if a point cloud dataset includes a central cluster of data points of one color, corresponding to the occupant's body, and an outer cluster of data points of a different color, representing the surrounding seating area, the processor 140 may determine that a driver is present in the occupant area.

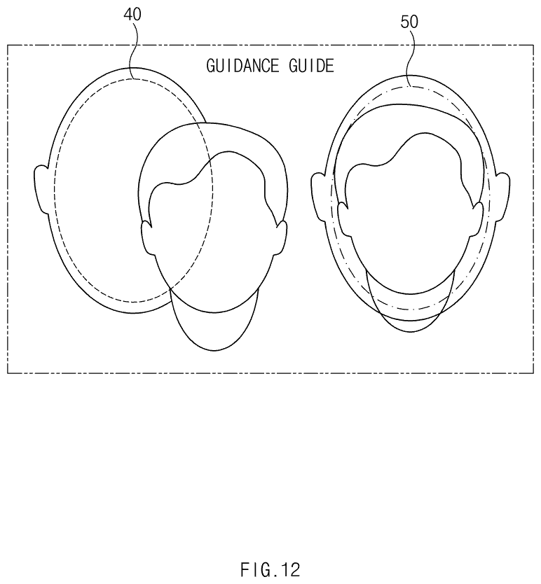

To prevent a non-driver other than the driver from taking the alcohol test, the processor 140 may output a guide (e.g., an on-screen positioning guide), ensuring that the driver's face is positioned in the center of the designated occupant area. The processor 140 may output a message or image (e.g., a visual indicator, a textual message, or an audio cue, etc.) indicating that the face is not centered, through the output device 130. Detailed descriptions refer to FIG. 12.

FIG. 12 shows an example of a guidance guide output according to an example of the present disclosure.

As shown in FIG. 12, the processor 140 may output a face image, which is captured by using a camera system (e.g., an RGB camera, an infrared facial recognition sensor, or a depth-sensing module, etc.), and a guide line where the captured face needs to be located, through the output device 130. When a driver's face is misaligned (e.g., not located within the guide line), the processor 140 may output a red alarm line 40. When the driver's face is located within the guide line, the processor 140 may output a green alarm line 50, and may allow the driver to intuitively recognize whether the driver's face is located at the center of an occupant area. When the red alarm line 40 is output, the processor 140 may output a message for requesting a retry. If the red alarm line 40 is repeatedly triggered beyond a preset number of times, the processor 140 may control an ignition lock.

If the driver's door is open, the processor 140 may determine whether there is an occupant in the occupant area. If an occupant is detected, the processor 140 may allow the scanner 110 to be turned on.

If a start button is pressed, the processor 140 may allow the scanner 110 to scan the occupant area. According to an example, to prevent a non-driver other than the driver from taking the alcohol test, the processor 140 may output a guide such that the driver's face is positioned in the center of the occupant area. According to an example, the processor 140 may set the wavelength of the infrared light source such that the infrared light source is absorbable by an alcohol gas, determine the absorbance of the alcohol gas based on the transmittance characteristics of the infrared light source. The processor 140 may estimate the alcohol concentration for each zone based on the absorbance of the alcohol gas, and generate an alcohol concentration mapping image according to the estimated alcohol concentration.

The processor 140 may determine whether an occupant is in a drunken state, based on the alcohol concentration mapping image.

According to an example, if the occupant is not in a drunken state (e.g., not intoxicated), based on the alcohol concentration mapping image, the processor 140 may allow the ignition to be turned on. Here, the allowing of the ignition to be turned on may indicate an operation of supplying power to an electrical device within a vehicle for driving the vehicle. On the other hand, when the driver presses the start button (not the ignition button), this does not immediately start the vehicle's engine or other vehicle driving systems (e.g., including an electric motor system) but instead initiates the scanning process. If the scanning process is successfully completed (e.g., indicating that the driver is not intoxicated), the processor 140 then allows the ignition to turn on, enabling the vehicle to start.

If the occupant is in a drunken state based on an alcohol concentration mapping image, the processor 140 may determine a location of the occupant. According to an example, the processor 140 may compare the newly generated alcohol concentration mapping image with pre-stored reference mapping data (e.g., pre-stored alcohol concentration mapping images of the occupant of the drunken state), and determine a location of the occupant of the drunken state based on the comparison.

If the occupant in the drunken state is a driver, the processor 140 may control the ignition lock. If the occupant of the drunken state is a passenger or a second-row occupant, the processor 140 may allow the ignition to be turned on.

When the vehicle starts driving, the processor 140 may periodically re-determine a state of the occupant based on a predefined monitoring interval.

Upon a preset period expiring during driving, the processor 140 may allow the scanner 110 to newly scan the occupant area. According to an example, the processor 140 may set the wavelength of the infrared light source such that the infrared light source is absorbable by an alcohol gas. The processor 140 may determine the absorbance level of the alcohol gas based on the transmittance characteristics of the infrared light source, estimate the alcohol concentration level for each scanning zone based on the absorbance level of the alcohol gas. The processor 140 may generate an alcohol concentration mapping image according to the estimated alcohol concentration.

The processor 140 may determine whether an occupant is in a drunken state, based on the alcohol concentration mapping image.

According to an example, if the occupant is not in a drunken state (e.g., not intoxicated), based on the alcohol concentration mapping image, the processor 140 may allow driving to be maintained or continued.

If the occupant is in a drunken state based on an alcohol concentration mapping image, the processor 140 may determine a location of the occupant. According to an example, the processor 140 may compare the newly generated alcohol concentration mapping image with pre-stored intoxication mapping data (e.g., pre-stored alcohol concentration mapping images of the occupant of the drunken state) and determine a location of the occupant of the drunken state based on the comparison.

If the occupant in the drunken state is a driver, the processor 140 may allow driving to be restricted. For example, the processor 140 may gradually reduce speed, issue visual and audible warnings, and activate hazard lights to alert surrounding drivers. The processor 140 may also guide the driver to a safe stopping point while restricting throttle input to prevent aggressive driving or autonomously perform a minimum risk maneuver operation to stop the vehicle. Once stopped, the transmission may be locked, preventing further movement. If the driver remains unresponsive, the processor 140 may notify emergency contacts or authorities. Vehicles with ADAS may also provide enhanced steering and braking assistance to maintain control and ensure a safe stop.

If the occupant in the drunken state is a passenger or a second-row occupant, the processor 140 may allow driving to be maintained or continued.

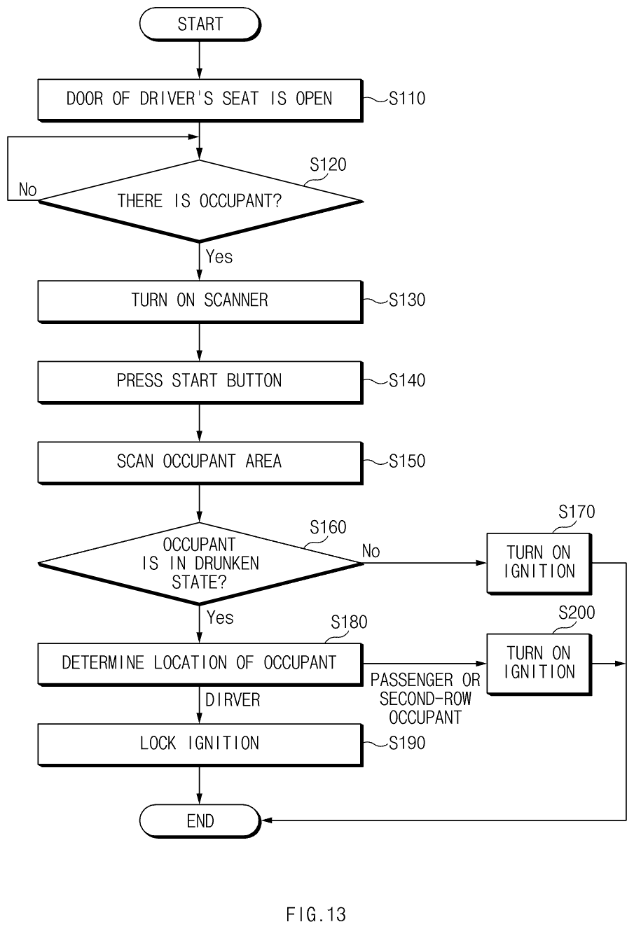

FIG. 13 shows an example of a method for determining a vehicle occupant state before driving, according to an example of the present disclosure.

As shown in FIG. 13, if a door of a driver's seat is open (S110), the processor 140 may determine whether there is an occupant in an occupant area (S120).

If the occupant is present in the occupant area, the processor 140 may allow the scanner 110 to be turned on (S130).

If a start button is pressed (S140), the processor 140 may allow the scanner 110 to scan the occupant area (S150). According to an example, to prevent a non-driver other than the driver from taking the alcohol test, the processor 140 may output a guide (e.g., a facial alignment guide), ensuring that the driver's face is positioned in the center of the occupant area. According to an example, the processor 140 may set the wavelength of the infrared light source such that the infrared light source is absorbable by an alcohol gas. The processor 140 may determine the absorbance level of the alcohol gas based on the transmittance characteristics of the infrared light source, and estimate the alcohol concentration level for each scanning zone based on the absorbance level of the alcohol gas. The processor 140 may generate an alcohol concentration mapping image according to the estimated alcohol concentration.

The processor 140 may determine whether the occupant is in a drunken state, based on the alcohol concentration mapping image (S160).

According to an example, if the occupant is not in a drunken state, based on the alcohol concentration mapping image, the processor 140 may allow the ignition to be turned on (S170). Here, the allowing of the ignition to be turned on may indicate an operation of supplying power to an electrical device within a vehicle for driving the vehicle. On the other hand, pressing the start button (not the ignition button), only allows to initiate the scanning process. If the scanning process is successfully completed (e.g., indicating that the driver is not intoxicated), the processor 140 then allows the ignition to turn on, enabling the vehicle to start to perform a driving operation.

If the occupant is in a drunken state based on an alcohol concentration mapping image, the processor 140 may determine a location of the occupant (S180). According to an example, the processor 140 may compare the newly generated alcohol concentration mapping image with pre-stored reference mapping data (e.g., pre-stored alcohol concentration mapping images of the occupant of the drunken state) and determine a location of the occupant of the drunken state based on the comparison (e.g., the newly generate image matching with one of the pre-stored alcohol concentration mapping images, for example, one of FIG. 7, FIG. 8, or FIG. 9).

If the occupant in the drunken state is a driver, the processor 140 may control the ignition lock (S190) (e.g., restricting the driver not to drive the vehicle). If the occupant of the drunken state is a passenger or a second-row occupant, the processor 140 may allow the ignition to be turned on (S200) enabling the vehicle to start driving.

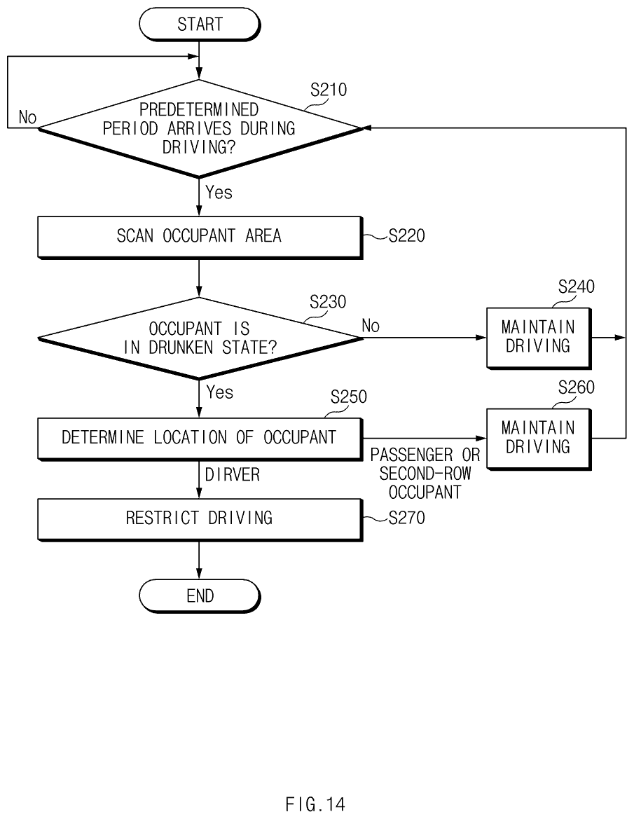

FIG. 14 shows an example of a method for determining a vehicle occupant state during driving, according to an example of the present disclosure.

When a vehicle starts driving, the processor 140 may periodically re-determine the drunken state of an occupant based on a predetermined period.

The processor 140 may determine whether the predetermined period expires during driving (S210).

If the predetermined period expires, the processor 140 may allow the scanner 110 to newly scan the occupant area (S220). According to an example, to prevent a non-driver other than the driver from taking the alcohol test, the processor 140 may output a guide, ensuring that the driver's face is positioned in the center of the occupant area. According to an example, the processor 140 may set the wavelength of the infrared light source such that the infrared light source is absorbable by an alcohol gas. The processor 140 may determine the absorbance level of the alcohol gas based on the transmittance characteristics of the infrared light source and estimate the alcohol concentration level for each scanning zone based on the absorbance level of the alcohol gas. The processor 140 may generate an alcohol concentration mapping image according to the estimated alcohol concentration level.

The processor 140 may determine whether the occupant is in a drunken state, based on the alcohol concentration mapping image (S230).

According to an example, if the occupant is not in a drunken state, based on the alcohol concentration mapping image, the processor 140 may allow driving to be maintained or continued (S240).

If the occupant is in a drunken state based on an alcohol concentration mapping image, the processor 140 may determine a location of the occupant (S250). According to an example, the processor 140 may compare the alcohol concentration mapping image with pre-stored reference mapping data (e.g., pre-stored alcohol concentration mapping images of the occupant of the drunken state) and determine a location of the occupant of the drunken state based on the comparison (e.g., the newly generate map matching with one of pre-store mapping images such as FIG. 7, FIG. 8, or FIG. 9).

If the occupant in the drunken state is a driver, the processor 140 may allow driving to be restricted (S270). For example, the processor 140 may reduce speed, issue warnings, activate hazard lights, guide to a safe stop, lock the transmission, and, if needed, notify emergency contacts or authorities while assisting with steering and braking. Alternatively or additionally, the processor 140 may autonomously perform a minimum risk maneuver operation to stop the vehicle. If the occupant in the drunken state is a passenger or a second-row occupant, the processor 140 may allow driving to be maintained (S260).

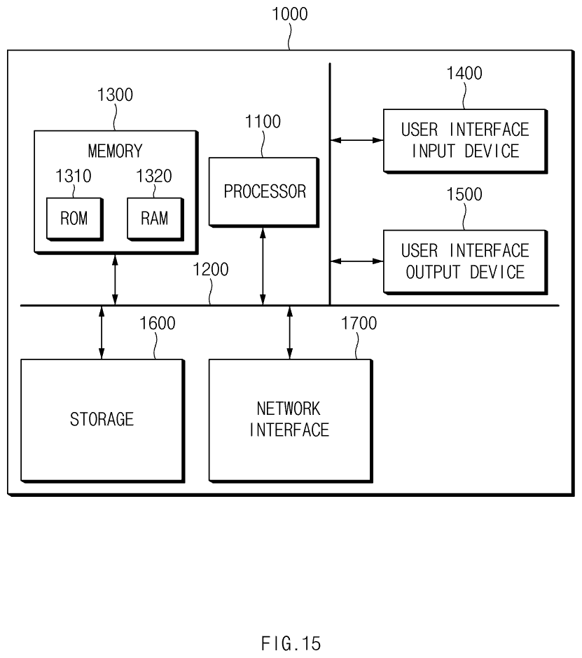

FIG. 15 shows an example of a configuration of a computing system performing a method, according to an example of the present disclosure.

Referring to FIG. 15, a computing system 1000 may include at least one processor 1100, a memory 1300, a user interface input device 1400, a user interface output device 1500, a storage 1600, and a network interface 1700, which are connected with each other via a bus 1200.

The processor 1100 may be a central processing unit (CPU) or a semiconductor device that processes instructions stored in the memory 1300 and/or the storage 1600. Each of the memory 1300 and the storage 1600 may include various types of volatile or nonvolatile storage media. For example, the memory 1300 may include a read only memory (ROM) 1310 and a random access memory (RAM) 1320.

Accordingly, the operations of the method or algorithm described in connection with examples disclosed in the specification may be directly implemented with a hardware module, a software module, or a combination of the hardware module and the software module, which is executed by the processor 1100. The software module may reside on a storage medium (i.e., the memory 1300 and/or the storage 1600) such as a random access memory (RAM), a flash memory, a read only memory (ROM), an erasable and programmable ROM (EPROM), an electrically EPROM (EEPROM), a register, a hard disk drive, a removable disc, or a compact disc-ROM (CD-ROM). The storage medium may be coupled to the processor 1100. The processor 1100 may read out information from the storage medium and may write information in the storage medium. Alternatively, the storage medium may be integrated with the processor 1100. The processor and storage medium may be implemented with an application specific integrated circuit (ASIC). The ASIC may be provided in a user terminal. Alternatively, the processor and storage medium may be implemented with separate components in the user terminal.

An example of the present disclosure provides an apparatus configured to determine a state of an occupant of a vehicle that may determine whether alcohol is detected, by using the property in which a specific gas absorbs a specific wavelength of an infrared light source, and a method thereof.

An example of the present disclosure provides an apparatus configured to determine a state of an occupant of a vehicle that may create an alcohol concentration mapping image by matching scan information of a driver's seat with the absorbance of an alcohol gas, and may determine whether the driver is in a drunken state, based on the alcohol concentration mapping image, and a method thereof.

An example of the present disclosure provides an apparatus configured to determine a state of an occupant of a vehicle that may determine the drunken state of the driver, a passenger, and a second-row occupant based on the alcohol concentration mapping image, and a method thereof.

An example of the present disclosure provides an apparatus configured to determine a state of an occupant of a vehicle that may enable quick starting by preventing delays due to the time that it takes to detect alcohol after boarding the vehicle by determining the drunken state, and a method thereof.

The technical problems to be solved by the present disclosure are not limited to the aforementioned problems, and any other technical problems not mentioned herein will be clearly understood from the following description by those skilled in the art to which the present disclosure pertains.

According to an example of the present disclosure, an apparatus configured to determine a state of an occupant of a vehicle may include a scanner that scans an occupant area by using an infrared light source absorbed by a specific gas, and a processor that generates a gas concentration mapping image based on absorbance of the specific gas, and determines a state of an occupant based on the gas concentration mapping image.

In an example, the specific gas may be included in exhalation of the occupant.

In an example, the processor may set a wavelength range of the infrared light source such that the infrared light source is absorbed by an alcohol gas.

In an example, the processor may calculate absorbance of the alcohol gas based on transmittance of the infrared light source passing through the alcohol gas.

In an example, the processor may generate an alcohol concentration mapping image based on the absorbance of the alcohol gas.

In an example, the processor may determine a drunken state of the occupant based on the alcohol concentration mapping image.

In an example, the processor may determine a location of the occupant based on the alcohol concentration mapping image when determining the occupant is in the drunken state.

In an example, the processor may allow an ignition to be locked before driving a vehicle, if the occupant is in the drunken state and is a driver, based on the alcohol concentration mapping image.

In an example, the processor may re-determine the drunken state of the occupant whenever a predetermined period arrives while the vehicle is driving.

In an example, the processor may allow driving to be restricted if the occupant is in the drunken state and is the driver, based on the alcohol concentration mapping image while the vehicle is driving.

According to an example of the present disclosure, a method for determining a vehicle occupant state may include scanning an occupant area by using an infrared light source absorbed by a specific gas and generating a gas concentration mapping image based on absorbance of the specific gas and determining a state of an occupant based on the gas concentration mapping image.

In an example, the specific gas may be included in exhalation of the occupant.

In an example, the method may further include setting a wavelength range of the infrared light source such that the infrared light source is absorbed by an alcohol gas.

In an example, the method may further include calculating absorbance of the alcohol gas based on transmittance of the infrared light source passing through the alcohol gas.

In an example, the method may further include generating an alcohol concentration mapping image based on the absorbance of the alcohol gas.

In an example, the method may further include determining a drunken state of the occupant based on the alcohol concentration mapping image.

In an example, the method may further include determining a location of the occupant based on the alcohol concentration mapping image when determining the occupant is in the drunken state.

In an example, the method may further include allowing an ignition to be locked before driving a vehicle, if the occupant is in the drunken state and is a driver, based on the alcohol concentration mapping image.

In an example, the method may further include re-determining the drunken state of the occupant whenever a predetermined period arrives while the vehicle is driving.

In an example, the method may further include allowing driving to be restricted if the occupant is in the drunken state and is the driver, based on the alcohol concentration mapping image while the vehicle is driving.

The above description is merely an example of the technical idea of the present disclosure, and various modifications and modifications may be made by one skilled in the art without departing from the essential characteristic of the present disclosure.

Accordingly, examples of the present disclosure are intended not to limit but to explain the technical idea of the present disclosure, and the scope and spirit of the present disclosure is not limited by the above examples. The scope of protection of the present disclosure should be construed by the attached claims, and all equivalents thereof should be construed as being included within the scope of the present disclosure.

According to an example of the present disclosure, an apparatus and a method for determining a vehicle occupant state may determine whether alcohol is detected, by using the property in which a specific gas absorbs a specific wavelength of an infrared light source.

According to an example of the present disclosure, an apparatus and a method for determining a vehicle occupant state may create an alcohol concentration mapping image by matching scan information of a driver's seat with the absorbance of an alcohol gas, and may determine whether the driver is in a drunken state, based on the alcohol concentration mapping image.

According to an example of the present disclosure, an apparatus and a method for determining a vehicle occupant state may determine the drunken state of the driver, a passenger, and a second-row occupant based on the alcohol concentration mapping image.

According to an example of the present disclosure, an apparatus and a method for determining a vehicle occupant state may enable quick starting by preventing delays due to the time that it takes to detect alcohol after boarding the vehicle by determining the drunken state.

Hereinabove, although the present disclosure was described with reference to examples and the accompanying drawings, the present disclosure is not limited thereto, but may be variously modified and altered by those skilled in the art to which the present disclosure pertains without departing from the spirit and scope of the present disclosure claimed in the following claims.

What is claimed:

1. An apparatus of a vehicle, the apparatus comprising:
a scanner configured to scan, using an infrared light source, an area of an occupant in the vehicle, wherein at least a portion of infrared light, emitted from the infrared light source, is absorbed by a specific gas in the vehicle; and
a processor configured to:
generate, based on the at least the portion of infrared light absorbed by the specific gas, a concentration indicator, wherein the concentration indicator indicates a concentration level of the specific gas;
determine, based on the concentration indicator, a state of the occupant; and
generate, based on the determined state of the occupant, a control signal configured to restrict at least one operation of the vehicle.

2. The apparatus of claim 1, wherein the specific gas is at least partially present in exhalation of the occupant.

3. The apparatus of claim 1, wherein the processor is configured to:
set a wavelength range of the infrared light source such that the at least the portion of infrared light is absorbed by the specific gas, and wherein the specific gas is an alcohol gas.

4. The apparatus of claim 3, wherein the processor is configured to:
determine, based on transmittance characteristics of the infrared light passing through the alcohol gas, an absorbance level of the alcohol gas.

5. The apparatus of claim 4, wherein the processor is configured to:
generate, based on the absorbance level of the alcohol gas, the concentration indicator.

6. The apparatus of claim 5, wherein the processor is configured to:
determine, based on the concentration indicator, that the occupant is in an intoxicated state.

7. The apparatus of claim 6, wherein the processor is configured to:
determine, based on the concentration indicator, a location of the occupant in the vehicle.

8. The apparatus of claim 7, wherein the processor is configured to:
determine, based on the location of the occupant in the vehicle, that the occupant is a driver of the vehicle; and allow an ignition of the vehicle to be locked such that
the occupant in the intoxicated state is prevented from driving the vehicle.

9. The apparatus of claim 5, wherein the processor is configured to:
based on a predetermined period expiring while the vehicle is driving, re-determine, based on a newly generated concentration indicator, a state of the occupant, wherein the newly generated concentration indicator indicates a concentration level of the alcohol gas after the predetermined period.

10. The apparatus of claim 9, wherein the processor is configured to:
based on the re-determined state of the occupant being in an intoxicated state and based on the occupant being a driver of the vehicle, perform a minimum risk maneuver operation to stop the vehicle.

11. A method performed by an apparatus of a vehicle, the method comprising:
scanning, using an infrared light source, an area of an occupant in the vehicle, wherein at least a portion of infrared light, emitted from the infrared light source, is absorbed by a specific gas in the vehicle;
generating, based on the at least the portion of infrared light absorbed by the specific gas, a concentration indicator, wherein the concentration indicator indicates a concentration level of the specific gas;
determining, based on the concentration indicator, a state of the occupant; and
generating, based on the determined state of the occupant, a control signal configured to restrict at least one operation of the vehicle.

12. The method of claim 11, wherein the specific gas is at least partially present in exhalation of the occupant.

13. The method of claim 11, further comprising:
setting a wavelength range of the infrared light source such that the at least the portion of infrared light is absorbed by the specific gas, and wherein the specific gas is an alcohol gas.

14. The method of claim 13, further comprising:
determining, based on transmittance characteristics of the infrared light passing through the alcohol gas, an absorbance level of the alcohol gas.

15. The method of claim 14, further comprising:
generating, based on the absorbance level of the alcohol gas, the concentration indicator.

16. The method of claim 15, further comprising:
determining, based on the concentration indicator, that the occupant is in an intoxicated state.

17. The method of claim 16, further comprising:
determining, based on the map, a location of the occupant in the vehicle.

18. The method of claim 17, further comprising:
determining, based on the location of the occupant in the vehicle, that the occupant is a driver of the vehicle; and
allowing an ignition of the vehicle to be locked such that the occupant in the intoxicated state is prevented from driving the vehicle.

19. The method of claim 15, further comprising:
while the vehicle is driving, determining, based on a newly generated concentration indicator, a second state of the occupant, wherein the newly generated concentration indicator indicates a concentration level of the specific gas exceeding a threshold; and
performing, based on the second state of the occupant and based on the occupant being a driver of the vehicle, a minimum risk maneuver operation to stop the vehicle.

20. A method performed by an apparatus of a vehicle, the method comprising:
emitting light toward a designated area within the vehicle, wherein at least a portion of the light is absorbable by a targeted gas;
indicating, based on the at least the portion of the light being absorbed by the targeted gas, a concentration level of the targeted gas;
determining, based on the indicating, a condition of an occupant associated with the designated area within the vehicle; and
controlling, based on the determined condition of the occupant, at least one operation of the vehicle.
